(19) United States
(12) Patent Application Publication
(10) Pub. No.: US 2014/0121602 A2
(43) Pub. Date: May 1, 2014

(54) INFUSION PUMP

(71) Applicants: Guang NING, Shanghai (CN); Maohe LIU, Shanghai (CN); Weiqing WANG, Shanghai (CN); Shouyue SUN, Shanghai (CN); Jingdong ZHANG, Shanghai (CN); Xiaomin HUANG, Shanghai (CN)

(72) Inventors: Guang NING, Shanghai (CN); Maohe LIU, Shanghai (CN); Weiqing WANG, Shanghai (CN); Shouyue SUN, Shanghai (CN); Jingdong ZHANG, Shanghai (CN); Xiaomin HUANG, Shanghai (CN)

(73) Assignees: SHANGHAI MICROPORT LIFESCIENCES CO., LTD., Shanghai (CN); RUIJIN HOSPITAL, SHANGHAI JIAOTONG UNIVERSITY SCHOOL OF MEDICINE, Shanghai (CN)

(21) Appl. No.: 13/823,620

(22) PCT Filed: Aug. 11, 2011
(86) PCT No.: PCT/CN2011/078299

§ 371 Date: Aug. 14, 2013

(30) Foreign Application Priority Data
Dec. 30, 2010 (CN) 201010615266.9

(51) Int. Cl. A61M 5/145; A61M 5/172

(52) U.S. Cl. CPC A61M 5/145; A61M 5/172
USPC 604/152

(57) ABSTRACT

An infusion pump comprises a housing (1); and a controller (3), a direct current motor (4), a transmission mechanism (6), a rotary encoder (5), and a liquid reservoir (7), which are located within the housing (1), wherein: a piston (71) is provided within the liquid reservoir (7); the transmission mechanism (6) has one end connected with the piston (71) and the other end connected with the direct current motor (4); the rotation shaft of the rotary encoder (5) is connected with the rotation shaft (41) of the direct current motor (4); the controller (3) is connected with the rotary encoder (5) and is provided thereon with a built-in clock and an input key (2) capable of receiving an external control signal. The infusion pump can freely adjust the infusion time and the infusion dose.

### TECHNICAL FIELD

The present application relates to the technical field of medical instruments, and in particular relates to a microinfusion pump.

### BACKGROUND ART

With respect to infertility patients caused by hypogonadotropic hypogonadism, Gonadotropin-Releasing Hormone (GnRH) or the like is generally adopted in clinic to be continuously injected into the patient's body to simulate a normal human body hormone level to treat the patient.

A micro pulse pump is a device which can achieve a continuous pulse infusion of GnRH and has a comparatively small impact on the patient's daily life and job at present, and is widely applied in clinic. The existing infusion manner of the micro pulse pump is generally to perform infusion regularly and quantitatively, i.e., to input quantitative drugs into the patient's body at a fixed time interval.

Upon study of the prior art, the applicant finds that since each patient has a different condition, the infusion time and the infusion volume should be adjusted according to the actual condition when the infusion is performed. However, the infusion time and the infusion volume at each time of the existing micro pulse pump are both preset, and cannot be freely adjusted in use, so the existing micro pulse pump cannot meet clinical requirements.

### SUMMARY OF THE INVENTION

In view of the above, the embodiments of the present application provide an infusion pump, which can freely control the number of turns of rotation of the direct current motor via a single chip microprocessor, and thus can freely adjust the infusion volume at each time, so that the infusion manner is more flexible.

In order to achieve the above object, the technical solutions provided by the embodiments of the present application are as follows:

An infusion pump, comprising

a housing, and

a controller, a direct current motor, a transmission mechanism, a rotary encoder, and a liquid reservoir, which are located within the housing, wherein:

one end of the liquid reservoir is provided with an outlet, and a piston is provided within the liquid reservoir;

the transmission mechanism has one end connected with the piston and the other end connected with the direct current motor, and the transmission mechanism can convert the rotary motion of the direct current motor into the linear motion of the piston;

a rotation shaft of the rotary encoder is connected with a rotation shaft of the direct current motor for controlling the number of turns of rotation of the direct current motor; and

the controller is connected with the rotary encoder and is provided thereon with a built-in clock and an input key capable of receiving an external control signal, and the controller calculates the number of turns of rotation of the direct current motor according to the received control signal, and sends the number of turns of rotation to the rotary encoder.

Preferably, the transmission mechanism comprises: a steering gear set, a screw rod, a screw nut and a push rod, wherein:

the screw rod is a hollow tube and has one end fixed to the piston;

the screw rod has one end connected with the direct current motor via the steering gear set and the other end penetrating into the push rod; and

the screw nut is fixed to the housing via a sliding groove, the screw on the screw nut matches the screw on the screw rod, and the screw nut is nested on the screw rod.

Preferably, the infusion pump further comprises: a storage connected with the controller for storing a control instruction received by the controller.

Preferably, the infusion pump further comprises: a screen connected with the controller for displaying a control instruction received by the controller.

Preferably, the controller is a single chip microprocessor.

It can be seen from the above technical solutions that in the infusion pump provided by the embodiments of the present application, a piston is provided within the liquid reservoir, the direct current motor is connected with the piston via the transmission mechanism, the transmission mechanism can convert the rotary motion of the direct current motor into the linear motion of the piston, the controller is connected with the direct current motor via the encoder, and an input key and a built-in clock are provided on the controller. Before using the infusion pump to perform the infusion, a drug solution is filled in the liquid reservoir, and the drug concentration, the liquid reservoir diameter, the transmission ratio of the transmission mechanism, the infusion time and the infusion dose are inputted via the input key on the controller; when the time of the built-in clock of the controller is equal to the infusion time, the controller calculates the number of turns the direct current motor needs to rotate and sends it to the rotary encoder; the rotary encoder controls the rotation of the direct current motor according to the received number of turns of rotation; and the direct current motor rotates to thereby drive the piston to move, and the motion distance of the piston is associated with the number of turns of rotation of the direct current motor.

Accordingly, the infusion pump provided by the embodiments of the present application can obtain the number of turns of rotation of the direct current motor by calculation according to the received control signal and control the rotation of the direct current motor according to the number of turns of rotation, and thus can freely control the infusion volume and can freely adjust the infusion time and the infusion dose with respect to patients of different conditions, thereby meeting requirements of the patients of different conditions.

### BRIEF DESCRIPTION OF THE DRAWINGS

In order to describe the embodiments of the present application or the technical solutions in the prior art more clearly, the figures to be used in the descriptions of the embodiments or the prior art will be briefly introduced below. It is obvious that the figures in the descriptions below are only some embodiments recorded in the present application, and those skilled in the art can also obtain other figures according to these figures without making inventive efforts.

FIG. 1 is an appearance schematic diagram of one infusion pump provided by the embodiments of the present application;

FIG. 2 is a structure schematic diagram of one infusion pump provided by the embodiments of the present application; and

FIG. 3 is a structure schematic diagram of another infusion pump provided by the embodiments of the present application.

### DETAILED DESCRIPTION

In order to make those skilled in the art better understand the technical solutions in the present application, clear and complete descriptions of the technical solutions in the embodiments of the present application will be given below by taking the figures in the embodiments of the present application into consideration. It is obvious that the described embodiments are only a part of the embodiments, rather than all the embodiments, of the present application. Based on the embodiments in the present application, all the other embodiments obtained by those skilled in the art without making inventive efforts shall belong to the scope of protection of the present application.

### Embodiment I

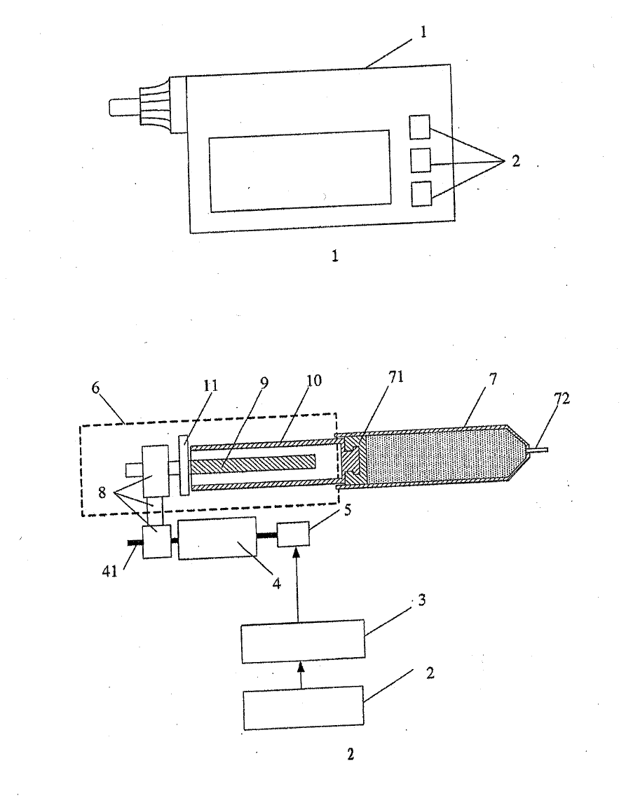

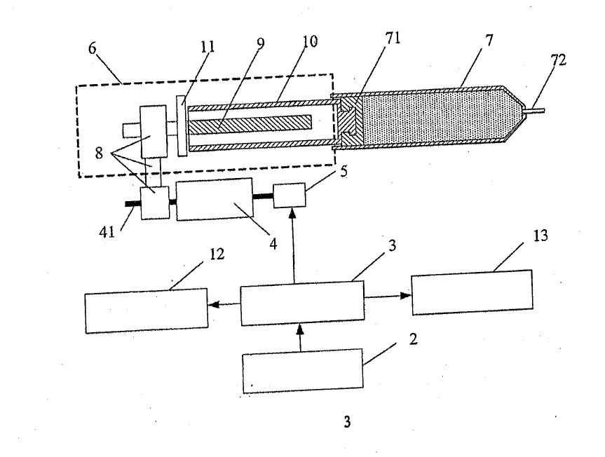

FIG. 1 is an appearance schematic diagram of one infusion pump provided by the embodiments of the present application. FIG. 2 is a structure schematic diagram of one infusion pump provided by the embodiments of the present application.

As shown in FIG. 1 and FIG. 2, the infusion pump comprises: a housing 1; and a controller 3, a direct current motor 4, a rotary encoder 5, a transmission mechanism 6, and a liquid reservoir 7, which are located within the housing 1, wherein: the controller 3 is electrically connected with the rotary encoder 5, the rotation shaft of the rotary encoder 5 is connected with the rotation shaft of the direct current motor 4, a piston 71 is provided within the liquid reservoir 7, and the direct current motor 4 is connected with the piston 71 in the liquid reservoir 7 via the transmission mechanism 6.

As shown in FIG. 2, the liquid reservoir 7 is of a tubular structure, one end of which is open and the other end of which is provided with an outlet 72. The piston 71 is provided within the liquid reservoir 7, and a seal is provided between the piston 71 and the inner wall of the liquid reservoir 7. A drug solution of a configured concentration can be placed in the liquid reservoir 7, the piston 71 moves from the open end of the liquid reservoir 7 to the outlet 72, and the drug solution within the liquid reservoir 7 can be discharged from the outlet 72.

As shown in FIG. 2, in the embodiments of the present application, the transmission mechanism 6 may comprise: a steering gear set 8, a screw rod 9, a screw nut 11 and a push rod 10. The steering gear set 8 is connected with the rotation shaft 41 of the direct current motor 4 and the screw rod 9, respectively, for transmitting the rotation motion of the rotation shaft 41 to the screw rod 9 to make the screw rod 9 rotate. The screw nut 11 is connected with the housing 1 via a sliding groove (not shown in the figure), and the sliding groove is arranged on the same line with the axis of the screw rod 9, i.e., the screw nut 11 can perform a linear motion on the housing 1; the screw on the screw nut 11 matches the screw on the screw rod 9, the screw nut 11 is nested on the screw rod 9, and when the screw rod 9 rotates, the screw nut 11 can be driven to move back and forth on the housing 1. The push rod 10 is a hollow tube, one end of which is fixed to the piston 71. The screw rod 9 penetrates into the push rod 10 through one end of the screw nut 11, and the other end of the push rod 10 contacts the screw nut 11, so when the screw rod 9 rotates and makes the screw nut 11 move in the direction toward the liquid reservoir 7, the screw nut 11 will push the push rod 10 to move, thereby pushing the piston 71 to move within the liquid reservoir 7. Thus, the transmission mechanism 6 just serves the purpose of converting the rotary motion of the direct current motor 4 into the linear motion of the piston 71, and the motion of the piston 71 in the direction toward the outlet 72 just discharges the drug solution within the liquid reservoir 7 via the outlet 72.

The rotary shaft of the rotary encoder 5 is connected with the rotary shaft 41 of the direct current motor 4 for controlling the number of turns of rotation of the direct current motor 4. In addition, the rotary encoder is further connected with the controller 3 for receiving the signal sent by the controller 3.

The controller 3 is provided thereon with an input key 2, and the controller 3 can receive a control signal inputted from the outside via the input key 2. The interior of the controller 3 is further provided with a built-in clock (not shown in the figure), and the time of the built-in clock accords with the actual date and time. The control signal inputted from the outside can be the infusion time, the input volume at each time and the drug concentration within the liquid reservoir. The controller 3 can be selected as a chip which can achieve its function and is well-known by those skilled in the art, and in the embodiments of the present application, the controller 3 preferably adopts a single chip microprocessor.

The infusion pump provided by the embodiments of the present application needs to set parameters such as the drug concentration, the liquid reservoir diameter and the transmission ratio of the transmission mechanism via the input key 2 beforehand before use. In use, the infusion time at each time and the infusion dose at each time are firstly inputted into the controller 3 via the input key 2; when the time of the built-in clock of the controller 3 is equal to the infusion time, the controller 3 calculates the number of turns the direct current motor 4 needs to rotate according to the infusion dose, the drug concentration, the liquid reservoir diameter and the transmission ratio of the transmission mechanism, and sends the number of turns of rotation to the rotary encoder 5; the rotary encoder 5 controls the direct current motor 4 according to the number of turns of rotation; the direct current motor 4 rotates to thereby drive the piston 71 to move via the transmission mechanism 6; and the piston 71 moves in the direction toward the outlet 72, i.e., the infusion can be performed. In addition, after the direct current motor rotates up to the above number of turns obtained by the calculation, the rotation is stopped and the infusion is stopped.

### Embodiment II

FIG. 3 is a structure schematic diagram of another infusion pump provided by the embodiments of the present application.

Based on the Embodiment I, as shown in FIG. 3, the infusion pump further comprises: a storage 12 mounted within the housing 1 and connected with the controller 3 for storing the control signal received by the controller 3. The storage 12 can store data such as the infusion time and the infusion dose to facilitate the patient to check the history infusion record.

Based on the Embodiment I, as shown in FIG. 3, the infusion pump further comprises: a screen 13 mounted on the housing 1 and connected with the controller 3 for displaying the control signal received by the controller 3. The screen 13, which is usually a liquid crystal screen, mainly serves the purpose of displaying the control signal inputted by the patient, so that it can prompt the patient to make modifications in time when the patient performs a wrong input.

It can be seen from the above technical solutions that in the infusion pump provided by the embodiments of the present application, a piston is provided within the liquid reservoir, the direct current motor is connected with the piston via the transmission mechanism, the transmission mechanism can convert the rotary motion of the direct current motor into the linear motion of the piston, the controller is connected with the direct current motor via the encoder, and an input key and a built-in clock are provided on the controller. Before using the infusion pump to perform the infusion, a drug solution is filled in the liquid reservoir, and the drug concentration, the liquid reservoir diameter, the transmission ratio of the transmission mechanism, the infusion time and the infusion dose are inputted via the input key on the controller; when the time of the built-in clock of the controller is equal to the infusion time, the controller calculates the number of turns the direct current motor needs to rotate and sends it to the rotary encoder; the rotary encoder controls the rotation of the direct current motor according to the received number of turns of rotation; and the direct current motor rotates to thereby drive the piston to move, and the motion distance of the piston is associated with the number of turns of rotation of the direct current motor.

Accordingly, the infusion pump provided by the embodiments of the present application can obtain the number of turns of rotation of the direct current motor by calculation according to the received control signal and control the rotation of the direct current motor according to the number of turns of rotation, and thus can freely control the infusion volume and can freely adjust the infusion time and the infusion dose with respect to patients of different conditions, thereby meeting requirements of the patients of different conditions.

The above contents are only preferred embodiments of the present application to enable those skilled in the art to understand or carry out the present application. A plurality of modifications of these embodiments are obvious to those skilled in the art, and general principles defined in this text can be carried out in other embodiments in the case of not breaking away from the sprint or scope of the present application. Thus, the present application will not be limited to these embodiments shown in this text, but shall accord with the widest scope consistent with the principles and novel characteristics disclosed in this text.

1. An infusion pump, characterized by comprising
a housing, and
a controller, a direct current motor, a transmission mechanism, a rotary encoder, and a liquid reservoir, which are located within the housing, wherein:
one end of the liquid reservoir is provided with an outlet, and a piston is provided within the liquid reservoir;
the transmission mechanism has one end connected with the piston and the other end connected with the direct current motor, and the transmission mechanism can convert the rotary motion of the direct current motor into the linear motion of the piston;
a rotation shaft of the rotary encoder is connected with a rotation shaft of the direct current motor for controlling the number of turns of rotation of the direct current motor; and
the controller is connected with the rotary encoder and is provided thereon with a built-in clock and an input key capable of receiving an external control signal, and the controller calculates the number of turns of rotation of the direct current motor according to the received control signal, and sends the number of turns of rotation to the rotary encoder.

2. The infusion pump according to claim 1, characterized in that the transmission mechanism comprises: a steering gear set, a screw rod, a screw nut and a push rod, wherein:
the screw rod is a hollow tube and has one end fixed to the piston;
the screw rod has one end connected with the direct current motor via the steering gear set and the other end penetrating into the push rod; and
the screw nut is fixed to the housing via a sliding groove, the screw on the screw nut matches the screw on the screw rod, and the screw nut is nested on the screw rod.

3. The infusion pump according to claim 1, characterized by further comprising: a storage connected with the controller for storing a control instruction received by the controller.

4. The infusion pump according to claim 1, characterized by further comprising: a screen connected with the controller for displaying a control instruction received by the controller.

5. The infusion pump according to claim 1, characterized in that the controller is a single chip microprocessor.
